(19) United States
(12) Patent Application Publication
(10) Pub. No.: US 2025/0066289 A1
(43) Pub. Date: Feb. 27, 2025

(54) BIGUANIDE DERIVATIVE OF IBUPROFEN AND PROCESS OF PREPARATION THEREOF

(71) Applicant: BVDU's Poona College of Pharmacy, Pune (IN)

(72) Inventors: Rajesh Rane, Pune (IN); Ratnadeep Patil, Pune (IN); Shreyash Moharir, Pune (IN); Dileep Kumar, Pune (IN); Prachi Khobragade, Pune (IN); Atmaram Pawar, Pune (IN)

(73) Assignee: BVDU's Poona College of Pharmacy, Pune (IN)

(21) Appl. No.: 18/387,857

(22) Filed: Nov. 8, 2023

(30) Foreign Application Priority Data
Jul. 19, 2023 (IN) 202321048702

(51) Int. Cl. C07C 279/26

(52) U.S. Cl. CPC C07C 279/26

(57) ABSTRACT

Disclosed is biguanide derivative of ibuprofen represented by the formula I,

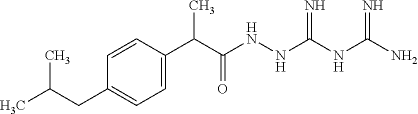 [SMILES: CC(C)Cc1ccc(C(C)C(=O)NNC(=N)NC(=N)N)cc1]

wherein the compound of formula I is N-carbamimidoyl-2-(2-(4-sobutylphenyl) propanoyl) hydrazine-1-carboximidamide. The biguanide derivative of ibuprofen of the present invention helps in significant reduction glucose levels in patients with diabetics as compared patients taking only metformin or similar biguanide. Further, the biguanide derivative of ibuprofen provides synergistic effect in lowering blood glucose level along with reducing inflammation in lower doses as compared to doses taken separately.

### FIELD OF THE INVENTION

The present invention relates to a biguanide derivative of ibuprofen and process for synthesis of the biguanide derivative of ibuprofen having purity of more than 97%.

### BACKGROUND OF THE INVENTION

The discovery of the blood-glucose-lowering capabilities of the herb Galega officinalis (French lilac) marked the beginning of the history of biguanides. Galegine, a guanidine derivative found in plant seeds and flowers, has been used to cure polyuria and other related disorders. Early in the 20th century, following the discovery of galegine, several biguanides (including synthelin A and B, biguanide, metformin, phenformin, and buformin) were developed, synthesized and further investigated for their antidiabetic properties, before being abandoned due to toxicity concerns or a presumption of low potency. The antihyperglycemic effects of these compounds are mostly due to their ability to block liver gluconeogenesis, which lowers blood glucose levels without increasing the risk of hypoglycemia in type 2 diabetes patients.

Biguanides are the class of compounds, which contains two guanidine moieties connected by a common nitrogen atom. These compounds have the property of being polar and hydrophilic molecules that are highly soluble in aqueous conditions due to the tautomerization of three amino and two imino groups. Metformin is one of the biguanide, chemically 3-(diaminomethylidene)-1,1-dimethylguanidine. Metformin's hypoglycemic activity in animal models was first reported by Slotta and Tschesche in 1929, and its clinical application was first described by Sterne in 1957.

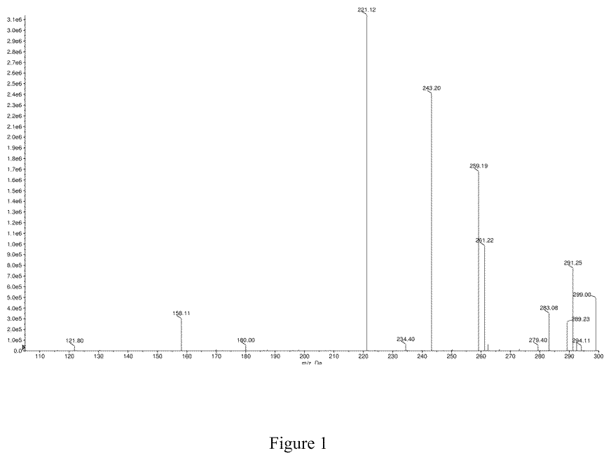

Ibuprofen is one of the most widely used analgesic, antipyretic, anti-inflammatory drug today. It is well established that ibuprofen is effective in controlling pain and inflammation in a variety of inflammatory and painful conditions. Among these are rheumatic and other musculoskeletal conditions, dental pain and surgery, dysmenorrhoea, upper respiratory tract conditions (colds, influenza), headaches, accidental sports injuries and surgical conditions. Basic molecular structure of Ibuprofen (1); Ibuprofen acid hydrazide (2); 2-Cyanoguanidine (3); and Biguanide of Ibuprofen (4), is shown in FIG. 1.

However, when these compounds, for example ibuprofen and metformin have to be administered separately, their dosage are higher as compared to their efficacy. Further, when the NSAIDs like ibuprofen are prescribed for long time, they pose various side effects such as swelling and the like. Specifically, these side effects are more when NSAIDs are administered to diabetic patients taking biguanides for diabetic treatment.

Accordingly, there exists a need to provide a pure form of a biguanide derivative of ibuprofen, which can be used as in treatment of various diseased conditions relating to pain and inflammation, and other skin related diseases involving pain and inflammation and which overcomes above mentioned drawbacks.

### OBJECT OF THE INVENTION

An object of the present invention is to decrease side effects of NSAIDs in diabetic patient taking biguanides.

Another object of the present invention is reduce pain and inflammation in patients with diabetic condition.

### DETAILED DESCRIPTION OF THE INVENTION

The foregoing objects of the present invention are accomplished and the problems and shortcomings associated with the prior art, techniques and approaches are overcome by the present invention as described below in the preferred embodiments.

The present invention provides a biguanide derivative of ibuprofen represented by the formula I,

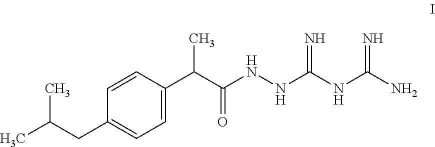 [SMILES: CC(C)Cc1ccc(C(C)C(=O)NNC(=N)NC(=N)N)cc1]

wherein the compound of formula I is N-carbamimidoyl-2-(2-(4-sobutylphenyl) propanoyl) hydrazine-1-carboximidamide.

The compound of formula I decreases side effects of NSAIDs in diabetic patient taking biguanides. Further, the compound of formula I reduces pain and inflammation in patients with diabetic condition.

In another aspect of the present invention a process for synthesis of biguanide derivative of ibuprofen. The process at first step comprises reacting ibuprofen with chloroform under anhydrous condition to form a first reaction mixture.

The process at second step comprises adding dimethylformamide (DMF) to the first reaction mixture to form a second reaction mixture.

The process at third step comprises adding thionyl chloride (SOCl2) to the second reaction mixture under nitrogen environment.

The process at fourth step comprises removing excess dichloromethane and SOCl₂ from the second reaction mixture by distillation.

The process at fifth step comprises adding petroleum ether to the second reaction mixture to obtain acid chloride of ibuprofen.

The process at sixth step comprises extracting, washing and drying the acid chloride of ibuprofen to obtain of 2-(4-isobutylphenyl) propanehydrazide. Specifically, the The acid chloride of ibuprofen is dissolved in dichloromethane followed by addition of triethylamine and hydrazine hydrate to obtain 2-(4-isobutylphenyl) propanehydrazide.

The process at seventh step comprises adding 4-isobutylphenyl) propanehydrazide an alcoholic solution followed by addition of dicyandiamide to form a third reaction mixture.

The process at final step comprises extracting, washing, and drying the third reaction mixture to obtain Ibuprofen biguanide monohydrochloride. Specifically, the ibuprofen biguanide monohydrochloride is N-carbamimidoyl-2-(2-(4-sobutylphenyl) propanoyl) hydrazine-1-carboximidamide as shown below.

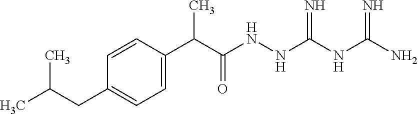 [SMILES: CC(C)Cc1ccc(C(C)C(=O)NNC(=N)NC(=N)N)cc1]

The ibuprofen biguanide monohydrochloride is recrystallized, dried under vacuum purified to obtain N-carbamimidoyl-2-(2-(4-isobutylphenyl) propanoyl) hydrazine-1-carboximidamide.

The scheme of the process for synthesis of the biguanide derivative of ibuprofen is shown below.

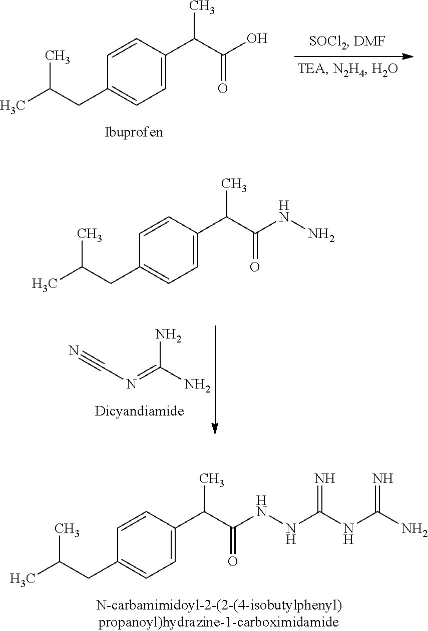 [SMILES: CC(C)Cc1ccc(C(C)C(=O)O)cc1 ; CC(C)Cc1ccc(C(C)C(=O)NN)cc1 ; N#CN=C(N)N ; CC(C)Cc1ccc(C(C)C(=O)NNC(=N)NC(=N)N)cc1]

### Example

### 1: Synthesis of 2-(4-isobutylphenyl) propanehydrazide

Under anhydrous condition, Ibuprofen (2.00 g, 0.096 mol) was dissolved in 100 mL of Chloroform and 2 drops of dry DMF was added into the vigorously stirred reaction mixture. SOCl2 (2.08 mL, 0.0288 mol) at 0° C. The complete addition of SOCl2 was done under Nitrogen. The reaction mixture was heated to 60° C. to reflux for 6 h to yield the yellow-colored liquid. Then after the completion of reaction the excess DCM and SOCl₂ was distilled out under reduced pressure. Then 20 mL petroleum ether was added to reflux for 15 min, and then the reaction mixture was cooled to room temperature and filtered out to obtain acid chloride of ibuprofen. The obtained crude product was dissolved in DCM and stirred for 10 mins. Then triethylamine (2.00 mL, 0.0144 mol TEA) was slowly added at 0° C. After addition of TEA the hydrazine hydrate (80%) (0.48, 0.0096 mol) was added dropwise in the reaction mixture and vigorously stirred for 30 min. The reaction progress was monitored by thin layer chromatography. After completion of reaction. The mixture was then poured into cold H₂O (50 mL) and extracted with CHCl₃ (3×25 mL each). The combined CHCl₃ layers were washed with H₂O (100 mL) and organic layer dried over Na₂SO₄ and then concentrated. The crude residue was washed and filtered with hexane (50 mL) to obtained the white solid powder as product and residual DMF and nonpolar impurities removed as filtrate. (72% yield); Melting point: 220-222° C.; MS m/z calculated for C₁₃H₂₀N₂O [M+H]: was 220.16; found: 221.12.

### 2: Synthesis of N-carbamimidoyl-2-(2-(4-isobutylphenyl) propanoyl) hydrazine-1-carboximidamide

In a round-bottomed flask equipped with a magnetic stirrer, accurately weighed compound 2 (1 g, 0.0 mol) was added in a round bottom flask containing a mixture of aq. HCl (1.10 mL, 0.0045 mol) and 50 mL absolute ethanol, and the mixture was stirred until it became homogeneous. Dicyandiamide (1.13 g, 0.0135 mol) was then added, and the mixture was heated at reflux with constant stirring for 12 h. The mixture was cooled to 0° C., and the gray solid powder was obtained after removing excess ethanol by distillation. The obtained crude product was washed with methanol to yield an initial crop of Ibuprofen biguanide monohydrochloride. The white powdered product was recrystallized from methanol, filtered, and finally dried under vacuum forming a white powder. The obtained crude compound was purified by column chromatography by using chloroform as eluent to give the desired product. (82% yield); Melting point: 372-374° C.; 1H NMR (400 MHZ, deuterium oxide): δ 0.88 (d, 6H), 1.30 (d, 3H), 1.82 (m, 1H), 2.43 (m, 2H), 3.53 (s, 1H), 8.20 (s, 4H); MS m/z calculated for C₁₅H₂₄N₆O [M+H]: was 304.20; found: 305.27; HPLC purity: 97.77%.

FIG. 1 shows MASS (+ve) graph,

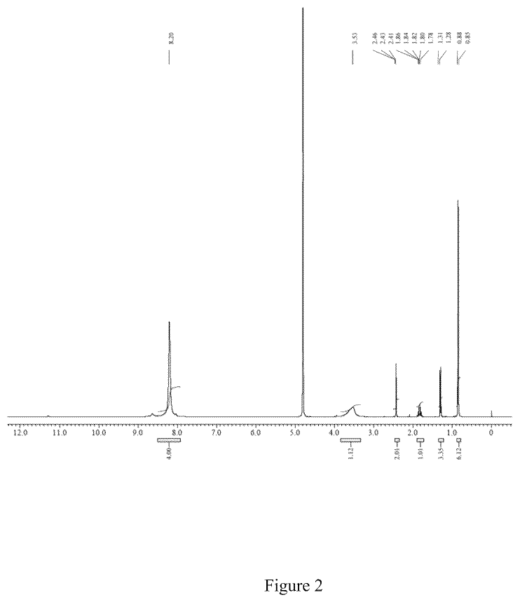

FIG. 2 shows H NMR graph,

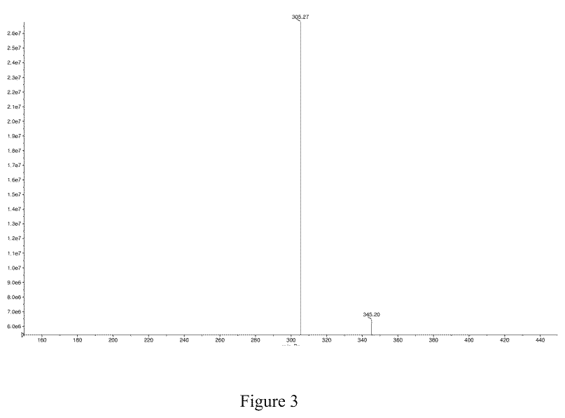

FIG. 3 shows Mass (+ve) and

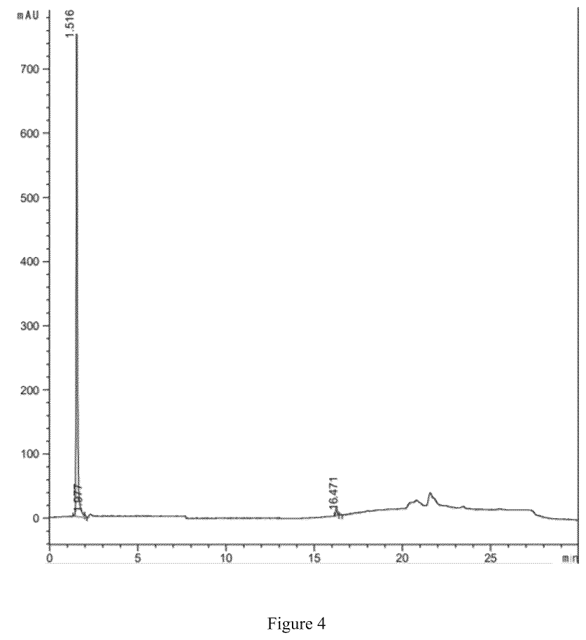

FIG. 4 shows HPLC graph of Ibuprofen biguanide monohydrochloride

### ANIMAL USE AND CARE

Male Wistar rats weighing between 200 and 250 g were used for the studies. The animals were kept in animal cages with free access to standard pellet food and water at a temperature of 23±2° C., a 12-hour cycle of darkness and light, and a relative humidity of roughly 45 to 65%. The Institutional Animal Ethics Committee (IAEC) authorized the protocol, and the animal experiments were carried out in accordance with it as well as the Indian regulations established by the Committee for the Purpose of Control and Supervision of Experimental Animals (CPCSEA), New Delhi.

### STZ Induced Diabetic Study for the Biguanide Derivative of Ibuprofen

An intraperitoneal injection of streptozotocin (STZ, 55 mg/kg of body weight) was used to cause diabetes. STZ was dissolved in 0.1 mol/L citrate buffer, pH 4.5, and injected into the peritoneal cavity. The fasting blood glucose concentration (FBG) was used for diagnosis of diabetes. Animals having blood glucose level greater than 16.7 mmol/L after 24 hours were deemed diabetic. These animals were further selected for the study.

The animals (n=4) were divided into four groups.
Group 1: treated with vehicle (distilled water) vehicle control;
Group 2: treated with vehicle (drinking water and STZ) diabetic control;
Group 3: treated with metformin (10 mg/kg body weight/day in drinking water) diabetic test; and
Group 4: treated with Biguanide of Ibuprofen (10 mg/kg body weight/day in drinking water) diabetic test.

The treatment with metformin and Biguanide of Ibuprofen was given for 14 days. And at the end of treatment fasting blood glucose estimation was performed.

TABLE 1
Effect of Biguanide of Ibuprofen of the present invention on
fasting blood glucose level (FBG) (Mean ± SD)

| | Fasting Blood | |
|---|---|---|
| | Glucose (mmol/L) | |
| Groups | Day 7 | Day 14 |
| Normal control | 5.93 ± 0.63 | 6.01 ± 0.60 |
| Diabetic Control | 23.21 ± 0.83 | 26.58 ± 1.41 |
| Diabetic + metformin | 22.77 ± 1.12 | 10.27 ± 1.21 |
| Diabetic + Biguanide of Ibuprofen | 22.93 ± 0.97 | 6.38 ± 0.86 |

### Effect of Biguanide of Ibuprofen of the Present Invention on Fasting Blood Glucose Level (FBG)

Biguanide of Ibuprofen demonstrated significant lowering of FBG levels. When compared to the control animal, the diabetic rat FBG levels were approximately four-fold elevated on day 7 of the treatment, and this rise persisted for 14 days. FBG levels in diabetic rats with Biguanide of Ibuprofen supplementation was significantly decreased. Biguanide of Ibuprofen produced more marked reduction in FBG when compared to metformin.

### CAR-Induced Anti-Inflammatory Study for the Biguanide Derivative of Ibuprofen

In the second experiment, the anti-inflammatory effect of Biguanide of Ibuprofen in comparison Ibuprofen was assessed by CAR-induced paw edema. The experimental groups were designed as follows (n=4 per group):
Group 1. Control group: 100 μl saline (i.d.)+saline (i.p.),
Group 2. CAR group: 100 μl CAR (i.d.)+saline (i.p.),
Group 3. Ibuprofen 5 mg/kg group: 100 μl CAR (i.d.)+5 mg/kg Ibuprofen (i.p.),
Group 4. Biguanide of Ibuprofen 5 mg/kg group: 100 μl CAR (i.d.)+5 mg/kg Biguanide of Ibuprofen (i.p.),

Carrageenan (100 μl) was injected by i.d. route per rat into the right hind paw. The rats were injected with i.p. Ibuprofen (5 mg/kg), and Biguanide of Ibuprofen (5 mg/kg) immediately after CAR injection. Saline was given to the control group. The paw volume was measured plethysmographically at 0 min, 30 min, 60 min, 90 min, 120 min, 180 min and 240 min. The 0th min reading was considered as the initial paw size of the rats. The change in the paw volume in the test groups was compared with the untreated control groups. The reduction in paw volume (mL) with the help of water plethysmometer was recorded.

TABLE 2
Mean and Standard Deviation of water volume displacement digital
plethysmometer (Mean ± SD)

| | | | Ibuprofen | Biguanide of |
|---|---|---|---|---|
| Time | Control | CAR group | 5 mg/kg | Ibuprofen |
| (min) | group (ml) | (ml) | group (ml) | 5 mg/kg group (ml) |
| 0 min | 0.686 ± 0.012 | 0.707 ± 0.012 | 0.700 ± 0.014 | 0.692 ± 0.012 |
| 30 min | 0.686 ± 0.012 | 0.833 ± 0.019 | 0.838 ± 0.021 | 0.853 ± 0.016 |
| 60 min | 0.688 ± 0.012 | 0.887 ± 0.020 | 0.897 ± 0.024 | 0.940 ± 0.013 |
| 90 min | 0.695 ± 0.012 | 0.950 ± 0.014 | 0.935 ± 0.024 | 0.875 ± 0.021 |
| 120 | 0.698 ± 0.012 | 1.013 ± 0.018 | 0.867 ± 0.012 | 0.815 ± 0.015 |
| min | | | | |
| 180 | 0.702 ± 0.008 | 1.112 ± 0.019 | 0.832 ± 0.008 | 0.740 ± 0.018 |
| min | | | | |
| 240 | 0.702 ± 0.008 | 1.203 ± 0.014 | 0.805 ± 0.014 | 0.710 ± 0.011 |
| min | | | | |

### The Effects of Biguanide of Ibuprofen on CAR-Induced Paw Edema

Paw volume did not have a significant difference between the test groups at all-time points, except the CAR group (Table 2). In the CAR group displaced volume of water which was 0.70 ml at 0 min rose to 1.20 ml at 240 min as shown in Table 2 showing there is increase in rat paw edema. Biguanide of Ibuprofen showed significantly decreased paw volume, when compared to that of the Ibuprofen. Results were expressed as mean±SD.

### Advantage of the Invention

1. Biguanide derivative of ibuprofen prepared by the process of the present invention helps in significant reduction glucose levels in patients with diabetics as compared patients taking only metformin or similar biguanide.

2. The biguanide derivative of ibuprofen of the present invention provides synergistic effect in lowering blood glucose level along with reducing inflammation in lower doses as compared to doses taken separately.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments were chosen and described in order to best explain the principles of the present invention and its practical application, and to thereby enable others skilled in the art to best utilize the present invention and various embodiments with various modifications as are suited to the particular use contemplated. It is understood that various omissions and substitutions of equivalents are contemplated as circumstances may suggest or render expedient, but such omissions and substitutions are intended to cover the application or implementation without departing from the spirit or scope of the claims of the present invention.

We claim:

1. A biguanide derivative of ibuprofen represented by the formula I, 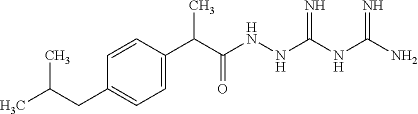 [SMILES: CC(C)Cc1ccc(C(C)C(=O)NNC(=N)NC(=N)N)cc1]
wherein the compound of formula I is N-carbamimidoyl-2-(2-(4-sobutylphenyl) propanoyl) hydrazine-1-carboximidamide.

2. A process for synthesis of biguanide derivative of ibuprofen, the process comprising:
reacting ibuprofen with chloroform under anhydrous condition to form a first reaction mixture;
adding dimethylformamide (DMF) to the first reaction mixture to form a second reaction mixture;
adding thionyl chloride (SOCl2) to the second reaction mixture under nitrogen environment;
removing excess dichloromethane and SOCl₂ from the second reaction mixture by distillation;
adding petroleum ether to the second reaction mixture to obtain acid chloride of ibuprofen;
extracting, washing and drying the acid chloride of ibuprofen to obtain of 2-(4-isobutylphenyl) propanehydrazide;
adding 4-isobutylphenyl) propanehydrazide an alcoholic solution followed by addition of dicyandiamide to form a third reaction mixture;
extracting, washing, and drying the third reaction mixture to obtain Ibuprofen biguanide monohydrochloride.

3. The process as claimed in claim 2, wherein the Ibuprofen biguanide monohydrochloride is N-carbamimidoyl-2-(2-(4-sobutylphenyl) propanoyl) hydrazine-1-carboximidamide.

4. The process as claimed in claim 2, wherein the acid chloride of ibuprofen is dissolved in dichloromethane followed by addition of triethylamine and hydrazine hydrate to obtain 2-(4-isobutylphenyl) propanehydrazide.

5. The process as claimed in claim 2, wherein the Ibuprofen biguanide monohydrochloride is recrystallized, dried under vacuum purified to obtain N-carbamimidoyl-2-(2-(4-isobutylphenyl) propanoyl) hydrazine-1-carboximidamide.
